(19) United States
(12) Patent Application Publication
(10) Pub. No.: US 2024/0066180 A1
(43) Pub. Date: Feb. 29, 2024

(54) Therapeutic Compositions Containing a Bitterant for Veterinary Wound Care

(71) Applicant: Keranetics, Inc., Winston-Salem, NC (US)

(72) Inventor: Luke Burnett, Winston-Salem, NC (US)

(73) Assignee: Keranetics, Inc., Winston-Salem, NC (US)

(21) Appl. No.: 18/453,663

(22) Filed: Aug. 22, 2023

Related U.S. Application Data
(60) Provisional application No. 63/373,356, filed on Aug. 24, 2022.

(51) Int. Cl. A61L 26/00

(52) U.S. Cl. CPC A61L 26/0066; A61L 26/008; A61L 2300/25; A61L 2300/802

(57) ABSTRACT

Described herein are therapeutic compositions comprising a wound healing agent such as keratin, and a bitterant, that can be used for skin care applications or to treat and heal wounds in veterinary medicine while preventing licking without the use of an Elizabethan collar.

This application claims benefit of U.S. Provisional Patent Application, Application No. 63/373,356, filed Aug. 24, 2022, the entire contents of which are fully incorporated herein by reference.

### 1. FIELD OF THE INVENTION

The invention relates to compositions of wound care products that contain bittering agents to prevent licking in veterinary wound and skin care applications.

### 2. BACKGROUND OF THE INVENTION

Wound healing is a dynamic process that requires the appropriate treatment and tissue environment and involves a complex interactions of cytokines, interleukins, growth factors and matrix proteins that are heavily influenced by local and systemic inflammatory mechanisms. There are many thousands of wound treatment products that are designed to provide a moist environment, promote angiogenesis and blood flow, maintain tissue temperature, provide protection against bacteria or contamination. Most of these products can generally be classified as “dressings”, which is a broad class of wound treatments that make direct contact with the injury. This is different than a bandage or secondary dressing, that is designed simply to keep the primary dressing in place and cover the wound.

Historically the techniques used to promote wound healing have not changed much in thousands of years and are largely based on using a variety of primary and secondary dressings, often known as wet-to-dry dressings. The earliest records of wound treatments from Egypt show that linens were commonly used after being soaked in oil or grease. In early Mesopotamia the addition of an occlusive plaster was added to keep the primary dressings in place. The Greeks often used wine or vinegar to clean a wound surface, followed by a treatment of honey to keep the wound moist. The understanding of antiseptic techniques to control infection was not introduced until the 19^{th} century and modern wound care techniques and treatments became widespread in the 20^{th} century.

The importance of closing a wound and keeping it moist and clean, is now known to be beneficial due to the maintenance of critical proteinases and growth-factors that are lost if the wound dries out or is exposed. Maintaining a moist wound environment promotes faster re-epithelization, collagen synthesis and an increase of hypoxia which decreases wound pH and helps reduce infection while increasing increase angiogenesis.

In human medicine, the importance of an optimal wound environment can be explained to the patient, as well as the need to use wound treatments that occlude the wound while maintaining a moist environment and keep the wound free of debris and contaminants. This is difficult in veterinary care with pets, as the patient's default self-care regimen for wounds is the use of oral lavage.

Dogs and cats sense of taste is not as developed as humans. Dogs have an estimated 1700 taste buds, and cats have only 480. This is significantly less than humans, and though both have receptors for bitter taste, they each largely rely on their sense of smell for taste deterrent. Thus, the appropriate bitterant to prevent licking must not just taste bitter, it must convey an odor to be a taste deterrent while not interfering with the normal wound healing process.

The earliest publications on veterinary wound care largely focused on farm and livestock animals, which are generally not physically able to access much of their body surface with their mouth. In pet medicine (i.e., dogs and cats), the usual treatment for wounds or surgical incisions, is to use a secondary dressing to occlude the wound, and place the animal in an Elizabethan collar to prevent licking or manipulation that can result in negative wound healing outcomes. These collars known as “e-collars” are highly disruptive to the animals normal daily routine, but are a necessary inconvenience to allow the normal wound healing processes to occur.

As noted, physical means of restraining behavior, e.g., Elizabethan collars, have drawbacks and may not provide reliable results. Additionally, it has been proposed that wound dressings be “treated” in some manner to discourage licking and chewing (see, e.g., US Pub. Pat. Appln. Pub. No. US 2012/0128805 A1). Herein, therapeutic compositions are provided that discourage biting and licking without additional steps or added compositions. In particular, they may be effectively used with keratin-based compositions.

Though there are several options that could be effective as a primary wound dressing in veterinary medicine, one of the most effective is keratin-based. Keratins are a family of proteins found in the hair, skin, and other tissues of vertebrates. Human hair is an important source of keratins because it is inexpensive and readily available. Although other sources of keratins are acceptable for the invention (e.g., hair, wool, fur, feathers, horns, hooves, nails, beaks, scales, and the like), human hair is optimal because of its biocompatibility and low antigenicity. Keratins are found in several FDA cleared medical wound care products, most notably KeraStat Gel and KeraStat Cream.

Keratins can be extracted from human hair fibers by oxidation or reduction. See, e.g., Crewther et al., The chemistry of keratins, in Advances in Protein Chemistry 20:191-346 (Christian B. Anfinsen, et al., eds., 1965) (hereafter “Crewther et al.”), which is incorporated by reference herein for the keratin extraction and purification methods and keratin biochemical, structural, and physicochemical properties disclosed therein. These processes were expanded in U.S. Pat. No. 10,385,095, “Methods for extracting keratin proteins,” fully incorporated wherein by reference. The extraction methods described in this issued patent comprise a two-step process whereby the crosslinked structure of hair keratins is broken down by either oxidation or reduction. In these reactions, the disulfide bonds formed by cystine amino acid residues are either oxidized or reduced, rendering the keratins soluble without disrupting the peptide bonds. After oxidation or reduction, keratins can remain trapped within the hair cuticle and often require a secondary denaturing step to efficiently extract the cortical proteins. These steps can be performed concomitantly for reductive extraction. Denaturants useful for such extractions include urea, guanidine salts, strong bases (hydroxides), detergents, and combinations thereof. A typical extraction method uses urea at concentrations between 0.1 and 10 M in buffered solutions.

Many protein purification techniques are known in the art, including fractional precipitation and immunoaffinity chromatography. See, e.g., Protein purification: Principles and practice, Springer (Robert K. Scopes, ed., 3rd ed. 1993); Protein purification techniques: A practical approach, Oxford University Press (Simon Roe, ed., 2nd ed. 2001); Isolation and purification of proteins, Marcel Dekker Inc. (Rajni Hatti-Kaul and Bo Mattiasson, eds., 2003), all of which are herein incorporated by reference for their disclosures related to protein purification. For example, sub-families of acidic and basic keratins have been described by Crewther et al. as being separable by moving boundary electrophoresis. See Crewther et al., supra.

The resulting solutions from dialysis (i.e., the permeate and retentate) are then separately lyophilized and ground with a mill into fine particulates that are several hundred micrometers in diameter. The alpha and gamma fractions can then be recombined as dry powders, sterilized via gamma radiation, and reconstituted with aqueous solutions such as water or phosphate buffered saline to produce keratose solutions or hydrogels.

The extraction methods described in U.S. Pat. No. 10,385,095 “Methods for extracting keratin proteins,” result in compositions of keratin gel that have significant advantages over what has been previously reported in the literature or in previous patents and are described in U.S. Pat. No. 9,700,631, “Low protein gelling compositions,” as well as U.S. Pat. No. 10,792,239, “White keratin compositions” both of which are also fully incorporated herein by reference.

Though the main embodiment described in this invention is a keratin-based wound healing product containing a bittering agent, there are many embodiments that could be used as the primary dressing. These include such hydrogels of natural polymers common in wound healing products such as alginate, cellulose, collagen, chitosan, chitin, fibroin, gelatin, honey, hyaluronic acid, silk, as well as self-assembling peptides and nanofibers; or keratins. There are also many synthetic materials that are used in various combinations for wound healing products comprise poly(lactic acid) (i.e., PLA), poly(glycolic acid) (i.e., PGA), poly(dioxanone) (i.e., PDS or PDO), poly(caprolactone), poly(PCCP-SA anhydride), polyoxyethylene (i.e., PEO), polyoxypropylene (i.e., PPO), poly(lactic-co-glycolic) acid (i.e., PLGA), polyethylene glycol (i.e., PEG), and poly (n-isopropylacrylamide) (i.e., PNIPAM). Other useful bioresorbable materials include poly(esters), poly(hydroxy acids), poly(lactones), poly(amides), poly(ester-amides), poly(amino acids), poly(anhydrides), poly(ortho-esters), poly(carbonates), poly(phosphazines), poly(thioesters), polysaccharides and mixtures thereof.

The therapeutic composition comprising a wound healing agent and a bitterant is anticipated to comprise various formulations of drugs, biomaterials, crosslinking agents, antibodies, biological tissues, artificial tissues, hydrogels, gels, membranes, films, scaffolds, sponges, beads, matrices, powders, particulates, absorbents, adsorbents, bandages, dressings, transdermal patches, coatings, creams, lotions, pastes, gels, jelly, mucilages, ointments, foams, sprays, aqueous or oily suspensions, emulsions, the like, or combinations thereof.

A wound healing agent may be formulated with a variety of bitterants including denatonium benzoate, bitter apple, bitter cherry, or vinegar.

### 3. SUMMARY OF THE INVENTION

Herein we describe an invention, where a therapeutic composition as a primary dressing designed to maintain wound moisture and facilitate wound healing, is impregnated with a bittering agent to form a composition that prevents an animal from licking or manipulating a wound.

Also described herein are wound healing compositions containing a bitterant delivered to a recipient in need thereof comprising a composition comprising drugs, biomaterials, crosslinking agents, antibodies, biological tissues, artificial tissues, hydrogels, gels, membranes, films, scaffolds, sponges, beads, matrices, powders, particulates, absorbents, adsorbents, bandages, dressings, transdermal patches, coatings, creams, lotions, pastes, gels, jelly, mucilages, ointments, foams, sprays, aqueous or oily suspensions, emulsions, the like, or combinations thereof.

The preferred embodiment described herein is a therapeutic composition comprising keratin where keratin consists of about 0.1%, about 0.2%, about 0.5%, about 0.75%, about 1%, about 2%, about 5%, about 10%, about 20%, about 30%, about 40%, about 50%, about 60%, about 70%, about 80%, about 90%, or about 100% of the composition by weight.

Also described herein are wound healing uses of compositions that contain a safe bitterant that does not impede wound healing such as denatonium benzoate. The preferred embodiment is a wound healing hydrogel containing a concentration of denatonium benzoate at a concentration of 0.5% (5000 ppm). Additional bitterants that may be used include sucrose octaacetate, quinine sulfate, naringen, and citronella. For bitterants with no odor of their own, an additional compound having a distinctive odor can be included to provide an olfactory signal to the animal to allow olfactory conditioning to occur and signal the presence of the bitterant by smell.

Additional therapeutic compositions comprising concentrations of bitterant are contemplated, and can contain bitterant at concentrations of about 0.0001, about 0.001, 0.01, 0.1, 0.2%, about 0.5%, about 0.75%, about 1%, about 2%, about 5%, about 10%, about 20%, about 30%, about 40%, about 50%, about 60%, about 70%, about 80%, about 90%, or about 100% of the composition by weight.

### 4. DETAILED DESCRIPTION OF THE INVENTION

### Brief Description of the FIGURES

FIG. 1 shows representative wound (spider bite) on the forelimb of a dog treated with a keratin biomaterial containing the bitterant denatonium benzoate. (A) is the wound at day 1, (B) is the wound at day 7, (C) is the wound at day 14, (D) is the wound at day 21.

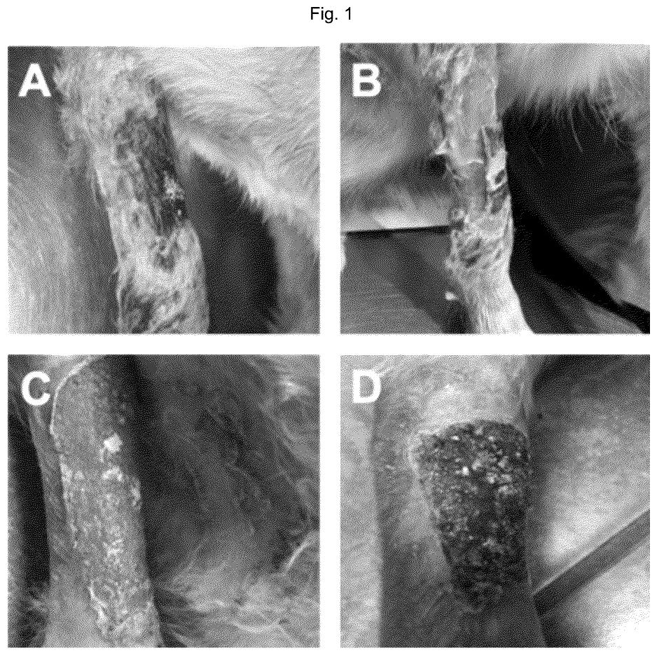

### TERMINOLOGY

The term “dressing” as used herein refers to a covering for a wound or injury. A dressing may comprise therapeutic compositions of the invention to modulate angiogenesis at the wound site or in surrounding tissue.

The phrase “topical application” as used herein refers to the administration of an agent to the surface of a body, tissue, or opening thereof, such as to skin, wounds, burns, site of surgery, or other injuries.

The term “keratin” as used herein refers to keratinous proteins that can be isolated from hair. Keratin may be derived from many sources; a typical source of keratin is human hair. Keratin can also be purified from wool, animal fur, feathers, horns, nails, or other keratinous structures. Keratins from wool also have generally good biocompatibility. See Ito et al., Biocompatibility of denatured wool keratin, Kobunshi Ronbunshu 39(4):249-256 (1982). The hair may be cleaned by washing in a warm water solution of mild detergent and freed of surface oils by washing with an organic solvent such as ethanol, ether, or acetone. A typical solvent is ethanol.

### DETAILED DESCRIPTION

Hydrogels comprising the keratin compositions containing bittering agents described herein are particularly useful for wound healing. Natural biomaterials useful for forming hydrogels for use in the compositions comprise alginate, cellulose, collagen, chitosan, chitin, fibroin, gelatin, honey, hyaluronic acid, silk, as well as self assemblying peptides and nanofibers; or keratins. Synthetic biomaterials useful in the compositions described herein comprise poly(lactic acid) (i.e., PLA), poly(glycolic acid) (i.e., PGA), poly(dioxanone) (i.e., PDS or PDO), poly(caprolactone), poly(PCCP-SA anhydride), polyoxyethylene (PEO), polyoxypropylene (PPO), poly(lactic-co-glycolic) acid (PLGA), polyethylene glycol (PEG), and poly (n-isopropylacrylamide) (PNIPAM). Other useful bioresorbable materials include poly(esters), poly(hydroxy acids), poly(lactones), poly(amides), poly(ester-amides), poly(amino acids), poly(anhydrides), poly(ortho-esters), poly(carbonates), poly(phosphazines), poly(thioesters), polysaccharides and mixtures thereof. Additional hydrogel forming compositions are described in U.S. Pat. No. 5,854,382.

Alternatively, the composition may comprise keratin in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94, 95%, 96%, 97%, 98%, 99%, or 100% by weight.

The composition may comprise a wound product with denatonium benzoate at concentrations of about 0.0001%, 0.001%, 0.01%, 0.1%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94, 95%, 96%, 97%, 98%, 99%, or 100% by weight

Also described herein are dry compositions comprising keratin. Such compositions may be in the form of powders, particulates, granules, beads, matrices or the like, and can be made in accordance with known techniques such as freeze drying (i.e., lyophilization), spray drying, dehydration, or the like.

The therapeutic compositions described herein may be produced by mixing dry powder compositions with aqueous solutions to produce the wound product. The mixing step can be carried out at any suitable temperature, typically room temperature, and can be carried out by any suitable technique such as stirring, shaking, agitation, sonication, etc. Salts, buffers, excipients, pharmaceutically acceptable carriers, gel-forming agents, disintegrants, coatings, anti-adherents, emulsifiers, detergents, reductants, oxidants, proteins, carbohydrates, or other constituent ingredients that may comprise a dry powder may also be included in the composition. The bitterant agent may be suspended in solutions, gels, or provided as dry powders. The compositions may be provided as hydrated gels, films, membranes, etc. or in dehydrated form. Compositions in dehydrated form may be provided with sterile or pyrogen-free water or buffer/electrolyte solutions for rehydrating.

Also described herein are therapeutic compositions comprising aqueous solutions, emulsions, or dry powders intended to be rehydrated with aqueous compositions or emulsions. Such compositions may contain proteins, carbohydrates, salts, buffers, excipients, pharmaceutically acceptable carriers, stabilizers, gel-forming agents, crowding agents, binders, coatings, solubilizers, emulsifiers, detergents, reductants, oxidants, preservatives, sorbents, polar or nonpolar organic solvents, non-aqueous, and aqueous solvents.

Also described herein are therapeutic compositions comprising delivery agents or carriers such as drugs, biomaterials, crosslinking agents, antibodies, biological tissues, artificial tissues, hydrogels, gels, membranes, films, scaffolds, sponges, beads, matrices, powders, absorbents, adsorbents, bandages, dressings, transdermal patches, coatings, creams, lotions, gels, pastes, jelly, mucilages, ointments, foams, sprays, aqueous or oily suspensions, or emulsions containing keratin and bitterant compositions. In addition, such substrates, agents, or delivery devises are useful for retaining a bitterant while promoting wound healing in the vicinity or periphery of a site of administration or at a specific tissue or tissues is contemplated. As such, administration of a keratin-bitterant composition as described herein may be spatially restricted and/or site specific.

The compositions described herein may be non-pyrogenic or sterile, they may be sterile filtered and processed aseptically, or terminally sterilized using ethylene oxide, e-beam, gamma radiation, or other low temperature methods (i.e., <50° C.).

The therapeutic compositions described herein may be provided preformed and aseptically packaged in suitable containers, such as foil tubes, flexible polymeric bags, plastic packaging, bottles, foil envelopes or may be provided as a kit of sterile or non-pyrogenic dry powder in one container and sterile aqueous solution in a separate container for mixing just prior to use. When provided pre-formed and packaged in a sterile container the composition preferably has a shelf life of at least 2 years at room temperature, prior to substantial loss of viscosity (e.g., more than 10 or 20 percent) and/or structural integrity of the gel or hydrogel.

### 5. EXAMPLES

### Example 1: Labeling Instructions for KeraVet Gel

KeraVet Gel (Rx only—wound Dressing by Keranetics, Inc., Winston-Salem, NC) is a non-implantable, water-based gelatinous (hydrogel) wound dressing intended to act as a protective covering in the management of a variety of partial thickness dermal wounds. KeraVet Gel is provided in a screw top multi-use tube. Each tube contains no less than 7 mL of KeraVet Gel, which contains keratin protein and is available by prescription from a veterinary practitioner.

### Indications for Use

KeraVet Gel is intended to provide a moist wound environment, absorb excess exudate, and deter animal licking behavior. KeraVet Gel is indicated for management of a number of partial thickness skin wounds such as: partial thickness (first and second degree) burns, severe sunburns, superficial injuries, cuts, abrasions, and incisions/surgical wounds. Under the direction of a veterinary practitioner, KeraVet Gel also may be used in the management of dry, light, and moderately exuding partial thickness wounds including: pressure (stage I-II) ulcers, venous stasis ulcers, ulcers caused by mixed vascular etiologies, diabetic ulcers, donor sites, and grafts. KeraVet Gel is not indicated for full thickness (third degree) burns.

### Warnings/Precautions

KeraVet Gel should not be used on bleeding wounds until the bleeding has been stopped.

Contact a veterinary professional if (1) signs of infection occur, (2) there is a change in wound color and/or odor, (3) the wound does not begin to show signs of improvement, or (4) any other unexpected symptoms occur.

KeraVet Gel is for external use only and should not be ingested. It should not be used for ophthalmic use. Keep this and all similar products out of reach of children.

### Directions for Use

1. Prepare wound area to ensure wound is free of debris. The wound may be gently flushed with a mild cleanser, if needed.
2. Inspect the KeraVet Gel tube to ensure that it has not been damaged or is past its expiration date. Remove the internal seal on the KeraVet Gel tube if opening the tube for the first time.
3. Apply KeraVet Gel to form a thin layer over the wound surface and edges of normal adjoining skin. Ensure the entire wound surface is covered with the hydrogel.
4. After application, a non-adherent, occlusive secondary dressing may be applied to maintain a moist environment and prevent removal of the hydrogel.

### Recommended Care

The dressing may be changed every 1 to 3 days, or as directed by a veterinary practitioner. When changing the dressing, lightly flush the wound bed with a gentle cleanser as determined by the veterinary practitioner and then apply KeraVet Gel by fully covering the wound and applying a secondary occlusive dressing, if needed. KeraVet Gel can be reapplied every 1 to 3 days for up to 30 days. If wound appearance does not improve within 30 days, consult a veterinary practitioner.

### Package Content

Each package contains one multi-use tube filled with no less than 7 mL of KeraVet Gel.

### Ingredients

Purified water, keratin, phenoxyethanol, carbomer, ethylhexylglycerin, sodium hydroxide, and denatonium benzoate

### Storage

KeraVet Gel should be stored in dry conditions at 59-86° F. (15-30° C.).
Do not use contents of KeraVet Gel if the tube appears damaged or is past the expiration date.
   KeraVet® is a registered trademark, and the product incorporates patented and/or patent pending technologies owned by KeraNetics, Inc.

We claim:

1. A therapeutic composition for healing wounds in animals, the composition comprising a wound healing agent and a bitterant to prevent licking.

2. The therapeutic composition of claim 1, wherein the wound healing agent is keratin.

3. The therapeutic composition of claim 1, where the bitterant is denatonium benzoate.

4. The therapeutic composition of claim 2 wherein the keratin comprises about 0.1%, about 0.2%, about 0.5%, about 0.75%, about 1%, about 2%, about 5%, about 10%, about 20%, about 30%, about 40%, about 50%, about 60%, about 70%, about 80%, about 90%, or about 100% of the composition by weight.

5. The therapeutic composition of claim 1, wherein the wound healing agent comprises hydrogels, gels, membranes, films, scaffolds, sponges, beads, matrices, powders, particulates, absorbents, adsorbents, bandages, dressings, transdermal patches, coatings, creams, lotions, pastes, gels, jelly, mucilages, ointments, foams, sprays, aqueous or oily suspensions, emulsions, or combinations thereof.

6. The therapeutic composition of claim 5, comprising a hydrogel of polymers selected from the group consisting of alginate, cellulose, collagen, chitosan, chitin, fibroin, gelatin, honey, hyaluronic acid, silk, self-assembling peptides, nanofibers, and combinations thereof.

7. The therapeutic composition of claim 1, comprising a synthetic material selected from the group consisting of poly(lactic acid) (i.e., PLA), poly(glycolic acid) (i.e., PGA), poly(dioxanone) (i.e., PDS or PDO), poly(caprolactone), poly(PCCP-SA anhydride), polyoxyethylene (PEO), polyoxypropylene (PPO), poly(lactic-co-glycolic) acid (PLGA), polyethylene glycol (PEG), and poly (n-isopropylacrylamide) (PNIPAM). Other useful bioresorbable materials include poly(esters), poly(hydroxy acids), poly(lactones), poly(amides), poly(ester-amides), poly(amino acids), poly(anhydrides), poly(ortho-esters), poly(carbonates), poly(phosphazines), poly(thioesters), polysaccharides, and mixtures thereof.

7. The therapeutic composition of claim 1, wherein the bitterant comprises denatonium benzoate, bitter apple, bitter cherry, or vinegar.

8. The therapeutic composition of claim 1, further comprising an odorant.

9. The therapeutic composition of claim 8, wherein the odorant is a citrus-based odorant.

10. A method of treating a wound in animals, comprising administering any of the compositions of any of the foregoing claims to the wound.
